(19) United States
(12) Patent Application Publication
(10) Pub. No.: US 2009/0137633 A1
(43) Pub. Date: May 28, 2009

(54) Stable pharmaceutical composition of rabeprazole

(76) Inventors: Bakulesh Mafatlal Khamar, Ahmedabad (IN); Jitendra Mohansingh Baweja, Gujarat (IN); Indravadan Ambalal Modi, Ahmedabad (IN); Alma Srinivas Reddy, Gujarat (IN)

Correspondence Address: DARBY & DARBY P.C., P.O. BOX 770, Church Street Station, New York, NY, 10008-0770, US

(21) Appl. No.: 10/568,747

(22) PCT Filed: Aug. 2, 2004
(86) PCT No.: PCT/IB04/02571

§ 371 Date: Dec. 19, 2007

(30) Foreign Application Priority Data
Aug. 18, 2003 (IN) 818/MUM/2003

(51) Int. Cl. A61K 31/435

(52) U.S. Cl. 514/338

(57) ABSTRACT

The present invention relates to a method of preparing a stable pharmaceutical composition of rabeprazole. The preparation may be used as an injectable dosage form in the treatment of severe gastric ulcers.

### FIELD OF THE INVENTION

The present invention relates to a method of preparing a stable rabeprazole pharmaceutical preparation, which gives a solution on reconstitution. The preparation can be used as an injectable preparation. The pharmaceutical composition of this invention finds application as an antiulcer activity.

### BACKGROUND OF THE INVENTION

Benzimidazole derivatives like omeprazole, pantoprazole, rabeprazole and lansoprazole belongs to a class of antisecretory compounds called proton pump inhibitors that do not exhibit anti-cholinergic or histamine H₂ receptor antagonist properties. Drugs of this class suppress gastric acid secretion by inhibiting the gastric H⁺ K⁺ ATPase enzyme system (proton pump) at the secretory surface of the gastric parietal cell. These class of drugs are commonly useful in the prevention and treatment of gastric related diseases, including reflux oesophagitis, gastritis, duodenitis, gastric ulcer and duodenal ulcer. The pharmaceutical compositions of these benzimidazole drugs utilize one or the other means to prevent drug degradation during its shelf life because the benzimidazoles in general, are acid labile drugs and have poor stability in aqueous solution.

Of the benzimidazole derivatives like omeprazole, pantoprazole, lansoprazole, and rabeprazole differs in its presence of 3-methoxy propoxy side chain. Rabeprazole sodium is chemically 2-{[[4-(3-methoxypropoxy)-3-methyl]-2-pyridinyl] sulfinyl}-1H-benzimidazole sodium salt. Christopher et al., (Drugs, 2001; 61 (15); 2327-2356), reports that rabeprazole has greater antisecretory effect over a 24 hr period than other benzimidazoles. It has duration of action ≧ to 24 hrs. The effect of rabeprazole on intragastric pH is unaffected by cytochrome P450 2C19 genotype, unlike omeprazole and lanzoprazole. The t-max is independent of dose and ranges between 2 and 5 hours and the oral bioavalability is about 50%. This leads to a requirement of injectable dosage form of rabeprazole for faster onset of action and increased bioavailability. Rabeprazole undergoes significant degradation in the aqueous solution. It is also reported that the stability of rabeprazole sodium is a function of pH. Aqueous instability of rabeprazole suggests the need for developing the parenteral preparation in lyophilized form, to be reconstituted at the time of administration.

U.S. Pat. No. 5,385,739, U.S. Pat. No. 6,159,499, U.S. Pat. No. 6,489,346, and U.S. Pat. No. 6,586,004 disclose stable pharmaceutical composition of benzimidazoles for use in solid dosage forms, and are not amenable to be applied as injectables.

Patent No DE 432-4014 describes a process for the production of a lyophilized form of pantoprazole sodium sesquihydrate. The said preparation contains, aqueous solutions of pantoprazole sodium sesquihydrate lyophilized in the presence of sucrose, as aid, at a temperature of −25° C. to −30° C. However, rabeprazole, when lyophilized similarly with sucrose, does not give stable product. The lyophilized product changes colour associated with loss of active and increase in concentration of degraded products.

Michel J. Akers, in his review article in J. Pharm Sci, Vol 91, No 11, 2002, p 2283-2300, has presented examples of synergistic and agonistic interactions that have been reported for excipients used in parenteral formulations. It has been reported that freeze-dried formulations typically contain one or more bulking agents like mannitol, lactose, sucrose, trehalose, dextran 40, and povidone. The moisture uptake behavior of these bulking agents both before and after freeze-drying has been discussed and the tendency for moisture uptake has been identified as a dominant factor to be considered in the development of formulations that are stable when freeze-dried. Mannitol is recommended to be widely used because of its low moisture uptake and crystallization tendency. The use of lactose has been specifically discouraged because of its relatively higher tendency for moisture uptake. It has been stated that addition of lactose destabilized the product since it does not allow crystallization. Lactose is amorphous after lyophilisation and gets converted to crystalline form after uptake of about 10% moisture, which may cause the product to degrade. It is further recommended that disaccharide carbohydrates like sucrose, trehalose alone do not result in storage stability of proteins, however addition of high-molecular weight carbohydrates such as dextran, which have high glass-transition temperature, stabilize protein preparations.

U.S. Pat. No. 5,536,735, discloses a pharmaceutical composition comprising a benzimidazole compound having anti-ulcer activity and a water-soluble carboxylic acid amide. According to the invention a water-insoluble benzimidazole compound having anti-ulcer activity can be solubilized by incorporation of carboxylic acid amide. The solid pharmaceutical composition as claimed in the invention can be extemporaneously dissolved in sterile distilled water or an infusion. Various sugar alcohols, when incorporated in the composition, act as form regulators and improve the morphology of the lyophilisate. For improving stability of the benzimidazole compound, a variety of salts and/or stabilizers like sodium citrate, sodium benzoate, magnesium carbonate, calcium carbonate etc. may be incorporated to the composition of this invention.

### OBJECTIVE OF THE INVENTION

The objective of the present invention is to prepare a stable pharmaceutical composition of rabeprazole, which provides an injectable dosage form. The product has faster onset of action and increase bioavailability.

### DETAILED DESCRIPTION OF THE INVENTION

The present invention relates to a method of preparing a stable pharmaceutical preparation of rabeprazole. The preparation can be used as an injectable dosage form. It is known that rabeprazole undergoes significant degradation in the aqueous solution; hence the need for developing the parenteral preparation in lyophilized form. The inventors carried out intensive studies to prepare a stable lyophilised rabeprazole preparation.

Lyophilisation of rabeprazole was done in conventional manner wherein the solution of rabeprazole in water for injection was filtered through 0.22-micron filter membrane, and lyophilized, wherein the freezing was done at −40° C., primary drying at −20 to −25° C. and secondary drying was done at 20 to 25° C. The resultant lyophilizate, thus obtained, was analyzed for residual moisture content, pH and clarity of reconstituted solution and assay by HPLC, and was found satisfactory. This preparation was stored in temperature humidity conditions of 2-8° C., 25° C./60% RH and 30° C./65% RH for studying its stability characteristics. A significant change in the physical characteristics of the preparation was observed at different time points at all storage conditions. This was also associated with loss of active ingredient and increase concentration of degradation products.

Lyophilisation of rabeprazole was done using sucrose as an aid, as described in Patent No DE 4324014. The resultant lyophilizate was found to be satisfactory in terms of its physicochemical properties. This product was subjected to stability studies. During the stability studies, the product was found to be degrading at all temperature and humidity conditions of storage at different time intervals within 2 months of studies.

Mannitol has been widely used as bulking agent because of its low moisture uptake tendency as suggested in the review article ‘Excipient-Drug Interactions in Parenteral Formulations’, of J. Pharm. Sci., Vol. 91, No. 11, p 2283′-2300. It is also preferred due to its crystallization tendency. Drug and mannitol were dissolved in the pyrogen free water. The solution was filtered through 0.22-micron membrane filter and lyophilized as described earlier. The lyophilizate was subjected to stability studies. It was observed that there was a significant change in colour of the product and it did not give a clear solution on reconstitution. This change was associated with degradation of drug and increase in the concentration of degradation products, at all storage conditions in different time frames within 2 months of studies.

As suggested in the same review article buffers, in varying ionic strengths, were incorporated in the solution of drug and mannitol, to stabilize the pH and thus prevent degradation of drug. Potassium dihydrogen phosphate and disodium hydrogen phosphate were added to the solution of drug and mannitol in pyrogen free water. The solution was then filtered and lyophilized. It was observed that the resultant lyophilizate was completely degraded and the reconstituted solution revealed presence of undissolved degraded drug in the form of black particles. Similar observations were made using carbonate buffer wherein sodium bicarbonate and sodium carbonate were incorporated in the solution of drug and mannitol. The resultant products were found to be degraded.

Antioxidants, in varying concentrations, were incorporated in the composition of solution containing drug and sugar alcohols to prevent oxidative degradation of drug during lyophilization. Sodium formaldehyde sulfoxylate was dissolved in the solution of drug and mannitol. The solution was filtered and lyophilized. The lyophilizate obtained was observed to be satisfactory with respect to the physical characteristics and the solution on reconstitution was clear and colourless. This product was subjected to stability studies at different temperature and humidity conditions. It was observed that after a period of 3 months the product stored at 30° C./65% RH and 25° C./60% RH, there was significant change in the physical characteristics of the product and the solution on reconstitution was also coloured and hazy. Loss of active drug was also observed and there was increase in the concentration of the degraded products. The sample stored at 2-8° C. was found to degrade within 6 months of the stability studies.

Combination of antioxidant and buffers like Sodium formaldehyde sulphoxylate, potassium dihydrogen phosphate, and disodium hydrogen phosphate was used. These excipients were dissolved in the solution of drug and mannitol in pyrogen free water. This solution was filtered and lyophilized. The resultant lyophilizate of this composition was completely degraded and the solution on reconstitution was found to contain degraded drug in the form of black particles. Sugar alcohol and/or antioxidant and buffers do not yield product with satisfactory stability characteristics

Other hexose based disaccharides as suggested were evaluated for their potential use as form regulators to prepare stable lyophilized composition of rabeprazole for parenteral administration. Glucose was dissolved along with the drug in pyrogen free water; solution was filtered through 0.22-micron membrane filter and lyophilized as described earlier. It was observed that the lyophilizate cake was not formed properly. So changes were made in the lyophilization cycle to increase the primary drying. The primary drying was done at −20° to −25° C. for period of 20 hours and the secondary drying was done at 20° C. to 25° C. for 12-14 hours. On changing the process there was improvement in the physical characteristics of the lyophilisate. The lyophilisate obtained was evaluated for its physicochemical properties. The product was found to be satisfactory and also the solution formed on reconstitution was clear and colourless. This product was subjected to stability studies as described earlier. It was observed that the product degraded at all the temperature and humidity conditions at different time intervals within 3 months.

Similarly incorporation of glucose, sucrose as bulking agents in varying concentrations in drug solution with or without buffer and/or in combination with antioxidant does not result in proper lyophilizate cake formation. The product developed color and the solution on reconstitution was found to be hazy, associated with loss of active drug. Surprisingly, lactose in appropriate range of concentrations when used as bulking agent with or without other excipients produced good lyophilisate cake in the vials. The solution on reconstitution was found to be clear. The product when subjected to stability studies at storage conditions of 2-8° C., 25° C./60% RH, and 30° C./65% RH was found to be stable on evaluation of all the parameters like reconstitution time, moisture content, HPLC potency and pH.

In accordance of this invention rabeprazole and lactose in an appropriate range of concentrations were dissolved in pyrogen free water. The resultant solution was filtered to make it sterile and fixed volume of this solution was filled in vials. These filled vials were lyophilized under controlled vacuum and temperature conditions in such a way that the temperature of product does not exceed −25° C. during primary drying stage and does not exceed 25° C. during secondary drying stage of lyophilization.

The invention is illustrated with a non-limiting example as below

### Example

22.5 gms of lactose was dissolved in 500 ml of pyrogen free water. To this solution 6.272 gms of rabeprazole sodium drug was added. The mixture was diluted with sufficient pyrogen free water to make 900 ml. This solution was sterilized by filtration through 0.22 micron bacterial filter and the filterate was distributed in 3 ml portion into 5 ml tubular glass vials. This solution was subjected to lyophilization whereby freezing was done at −40° C. Vacuum was fixed to a value of about 300 millitorr and condenser temperature kept at about 45° C. Primary drying was performed a −25° to −20° C. for 16 hours. Further the secondary drying was done at 20° to 25° for a period of 10 hours. The residual moisture content was kept in the range of 2-4%.

The resultant lyophilizate was subjected to stability studies and the results are shown in table 1.

The results reveal that the product obtained as above is stable when stored at 2-8° C., 25° C./60% RH and 30° C./65% RH. There is no significant change in the physical characteristics and the solution obtained on reconstitution of corresponding samples is clear and colorless.

To adjust the tonicity, agents like mannitol and sodium chloride may be added.

It is also observed that even when lactose is substituted with trehalose, galactose the stability of the composition is not affected in all of the above-mentioned examples.

| | |
|---|---|
| Rabeprazole Sodium AF | 20 mg |
| Lactose | 75 mg |

Summary of results: - (upto 6 months)

| | | | ASSAY (% w/v) | | |
|---|---|---|---|---|---|
| | Conditions | | Rabeprazole Sodium | | % |
| | &Period | Physical Observations | AF | pH | Moisture |
| | Releasing | Off white lyophilized | 18.0 mg to 22.0 mg | Between | NMT 8% |
| | Limits | powder, which on | (90% to 110%) | 8 to 11 | |
| | | reconstitution with 3 ml | | | |
| | | WFI gives clear solution. | | | |
| | Initial | Off white lyophilized | 19.49 mg (97.4%) | 9.64 | 3.73% |
| | | powder, which on | | | |
| | | reconstitution with 3 ml | | | |
| | | WFI gives clear solution. | | | |
| 25° C./ | 1 M | Same as initial | 19.06 mg (95.3%) | 9.52 | 3.91% |
| 60% RH | 2 M | Same as initial | 18.85 mg (94.2%) | 9.55 | 5.40% |
| | 3 M | Same as initial | 18.32 mg (91.6%) | 9.28 | 5.70% |
| | 6 M | Same as initial | 19.27 mg (96.3%) | 9.62 | 6.10% |
| 30° C./ | 1 M | Same as initial | 18.80 mg (94.0%) | 9.50 | 4.25% |
| 65% RH | 2 M | Same as initial | 18.47 mg (92.3%) | 9.57 | 5.48% |
| | 3 M | Same as initial | 18.77 mg (93.8%) | 9.20 | 6.03% |
| | 6 M | Same as initial | 18.49 mg (92.4%) | 9.59 | 6.33% |
| 2° C. to | 3 M | Same as intial | 19.64 mg (98.2%) | 9.45 | 5.21% |
| 8° C. | 6 M | Same as initial | 19.35 mg (96.7%) | 9.67 | 6.05% |

Other examples working successfully according to the present invention are mentioned below. These examples are not limiting to the scope of the invention.

### Working Compositions

### EXAMPLE-1

### Each Vial Contains

| | |
|---|---|
| Rabeprazole Sodium | 20 mg |
| Lactose | 75 mg |

### EXAMPLE-2

### Each Vial Contains

| | |
|---|---|
| Rabeprazole Sodium | 20 mg |
| Lactose | 60 mg |
| Galactose | 15 mg |

### EXAMPLE-3

### Each Vial Contains

| | |
|---|---|
| Rabeprazole Sodium | 20 mg |
| Lactose | 60 mg |
| Trehalose | 15 mg |

### EXAMPLE-4

### Each Vial Contains

| | |
|---|---|
| Rabeprazole Sodium | 20 mg |
| Lactose | 75 mg |
| Disodium hydrogen phosphate | 0.1 mg |

### EXAMPLE-5

### Each Vial Contains

| | |
|---|---|
| Rabeprazole Sodium | 20 mg |
| Lactose | 75 mg |
| Sodium carbonate decahydrate | 0.1 mg |

### EXAMPLE-6

### Each Vial Contains

| | |
|---|---|
| Rabeprazole Sodium | 20 mg |
| Lactose | 75 mg |
| Sodium sulfite | 0.1 mg |

### EXAMPLE-7

### Each Vial Contains

| | |
|---|---|
| Rabeprazole Sodium | 20 mg |
| Lactose | 60 mg |
| Mannitol | 15 mg |

### EXAMPLE-8

### Each Vial Contains

| | |
|---|---|
| Rabeprazole Sodium | 20 mg |
| Lactose | 45 mg |
| Trehalose | 30 mg |

### EXAMPLE-9

### Each Vial Contains

| | |
|---|---|
| Rabeprazole Sodium | 20 mg |
| Lactose | 60 mg |
| Sucrose | 15 mg |

Thus, it is apparent that there has been provided, in accordance with the instant invention, a process that fully satisfies the objects and advantages set forth herein above. While the invention has been described with respect to various specific examples and embodiments thereof, it is understood that the invention is not limited thereto and many alternatives, modifications and variations will be apparent to those skilled in the art in light of the forgoing description. Accordingly, it is intended to embrace all such alternatives, modifications and variations as fall with in the spirit and broad scope of invention.

1-9. (canceled)

10. A lyophilized pharmaceutical composition comprising 1% to 40% by weight of rabeprazole or a salt thereof, 55% to 99% by weight of lactose, galactose, trehalose or a combination thereof and 0% to 3% by weight of other excipients.

11. A lyophilized pharmaceutical composition as claimed in claim 1, comprising 1 to 30% by weight of rabeprazole or a salt thereof and 65-99% by weight of lactose, galactose, trehalose or a combination thereof.

12. The lyophilized pharmaceutical composition as claimed in claim 1 wherein the other excipients are selected from the group consisting of phosphate buffer, carbonate buffer, tonicity agents and antioxidants.

13. A therapy comprising delivering the lyophilized pharmaceutical composition as defined in claim 1.

14. A process for preparing an injectable dosage comprising dissolving the lyophilized pharmaceutical composition as defined in claim 1 in water.

15. A process for preparing a pharmaceutical composition comprising rabeprazole or a salt thereof, comprising:
a. dissolving rabeprazole or a salt thereof and lactose, galactose, trehalose or a combination thereof, with or without excipients in a solvent under stirring to form a solution;
b. adjusting the pH of the solution to 8.0-11.0
c. optionally removing any particulates from the solution; and
d. causing lyophilization of the solution.

16. The process as claimed in claim 6 wherein the solvent is water.

17. The process as claimed in claim 6 wherein the pharmaceutical composition contains at least 2 parts of lactose, galactose, trehalose or a combination thereof for one part of rabeprazole.

18. The process as claimed in claim 6 wherein said removing any particulates comprises filtering.

19. The process as claimed in claim 6 wherein lyophilization comprises primary drying at a product temperature below −10° C. and secondary drying at a temperature below 25° C.

20. The lyophilized pharmaceutical composition as claimed in claim 2 wherein the other excipients are selected from phosphate buffer, carbonate buffer, tonicity agents and antioxidants.

21. A therapy comprising delivering the lyophilized pharmaceutical composition as defined in claim 2.

22. A therapy comprising delivering the lyophilized pharmaceutical composition as defined in claim 3.

23. A process for preparing an injectable dosage comprising dissolving the lyophilized pharmaceutical composition as defined in claim 2 in water.

24. A process for preparing an injectable dosage comprising dissolving the lyophilized pharmaceutical composition as defined in claim 3 in water.

25. The process as claimed in claim 7 wherein the pharmaceutical composition contains at least 2 parts of lactose, galactose, trehalose or a combination thereof for one part of rabeprazole.

26. The process as claimed in claim 7 wherein said removing any particulates comprises filtering.

27. The process as claimed in claim 8 wherein said removing any particulates comprises filtering.

28. The process as claimed in claim 7 wherein lyophilization comprises primary drying at a product temperature below −10° C. and secondary drying at a temperature below 25° C.

29. The process as claimed in claim 8 wherein lyophilization comprises primary drying at a product temperature below −10° C. and secondary drying at a temperature below 25° C.
